SEQUENCE LISTING (1 sequence)

SEQ ID NO: 1 - protein, 74 aa, Homo sapiens
TKTESSSRGPYHPSECCFTYTTYKIPRQRIMDYYETNSQCSKPGIVFITKRGHSVCTNPSDKWVQDYIKDMKEN

(19) United States
(12) Patent Application Publication
(10) Pub. No.: US 2007/0244037 A1
(43) Pub. Date: Oct. 18, 2007

(54) Human Chemokine HCC-1 Polypeptides To Improve Stem Cell Transplantation

(75) Inventors: Rudolf Richter, Hannover (DE); Reinhard Hen-Schler, Frankfurt (DE); Wolf-Georg Forssmann, Hannover (DE)

Correspondence Address: JACOBSON HOLMAN PLLC, 400 SEVENTH STREET N.W., SUITE 600, WASHINGTON, DC, 20004, US

(73) Assignee: TAP PHARMACEUTICAL PRODUCTS INC, Lake Forest, IL (US)

(21) Appl. No.: 10/577,053

(22) PCT Filed: Oct. 16, 2004
(86) PCT No.: PCT/EP04/11691

§ 371 Date: May 9, 2007

(30) Foreign Application Priority Data
Oct. 24, 2003 (DE) 10349966.0
Feb. 19, 2004 (EP) 04003716.0

(51) Int. Cl. C07K 14/52; A61K 38/19; C07K 16/00; C12N 1/00; C12P 21/00

(52) U.S. Cl. 514/8; 435/243; 435/69.1; 530/322; 530/387.1; 530/388.1; 530/389.1

(57) ABSTRACT

The invention discloses the human chemokine HCC-1, N-terminally truncated HCC-1 molecules and glycosylated HCC-1 which improve the homing of stem cells into the -bone marrow during stem cell transplantation. It is also provided a procedure for producing the polypeptides by recombinant techniques or chemical synthesis and for producing antibodies against such polypeptide. Furthermore, it is disclosed the modification of the polypeptide by coupling of amino acid residues and/or chemical groups or deleting amino-acids generating potent derivatives of the polypeptide. Another aspect of the invention provides a combination of the polypeptide of the present invention and a suitable pharmaceutical carrier for providing a therapeutically effective amount of the polypeptide for the treatment of various associated diseases. The invention concerns also the use of the HCC-1 molecules to increase engraftment of stem cells in the course of the stem cell transplantation performed in stem cell transplantation related diseases.

### FIELD OF THE INVENTION

The present invention relates to methods of using the human chemokine HCC-1, N-terminally truncated HCC-1 and glycosylated HCC-1 to improve. stem cell homing into the bone marrow during stem cell transplantation.

### BACKGROUND OF THE INVENTION

Hematopoietic stem cells are rare primitive blood cell progenitors that have the capacity to self-replicate, to maintain a continuous source of regenerative cells, and to differentiate, to give rise to various morphologically recognizable precursors of blood cell lineages. These precursors are immature blood cells that cannot self-replicate and must differentiate into mature blood cells. Within the bone marrow microenvironment, the stem cells self-proliferate and actively maintain continuous production of all mature blood cell lineages throughout life.

Bone marrow transplantation is being increasingly used in humans as an effective therapy for an increasing number of diseases, including malignancies such as leukemias, lymphoma, myeloma and selected solid tumors as well as nonmalignant conditions such as aplastic anemias, immunological deficiencies and inborn errors of metabolism. The objective of BM transplantation is to provide the host with a healthy stem cell population that will differentiate into mature blood cells that replace deficient or pathologic cell lineages.

The source of the BM for transplantation may be autologous, syngeneic or allogeneic. Preferred are autologous BM or BM from HLA-matched siblings, but also BM from HLA-nonmatched donors is being used for transplantation.

Complicating factors in BM transplantation include graft rejection and graft-vs-host disease. Since donor T lymphocytes were found to cause GVHD in animals, one of the procedures to prevent or alleviate GVHD consists in removing T cells from the donor BM before transplantation. This can be done by different techniques. Extensive use of T-cell depleted BM effectively prevented GVHD but, unfortunately, resulted in a high rate of graft rejection (10-15% in HLA-matched recipients and 50% in HLA-nonmatched recipients) or graft failure (as high as 50%).

Another problem in BM transplantation is the difficulty of achieving long-term successful engraftment also when no graft rejection or GVHD occurs. Nowadays, patients which were successfully transplanted have very low levels of stem cells and immature progenitors which generate mature blood cells, compared with healthy individuals.

Stem cells are functionally defined by their ability to home to the bone marrow and to durably repopulate transplanted recipients with both myeloid and lymphoid cells. The processes that mediate homing and engraftment of human stem cells to the bone marrow involve a complex interplay between cytokines, chemokines and adhesion molecules.

Much of our knowledge of the regulation and the hierarchical organization of the hematopoietic system derives from studies in the mouse wherein stem cells' are identified and quantified in long-term reconstitution assays. In contrast, our knowledge of the biology of human hematopoiesis is limited, since it is mostly based on in characterize and quantify repopulating stem cells.

Intensive research is being carried out in order to understand the processes that mediate homing and engraftment of human stem cells to the bone marrow. Recently, several groups have established in vivo models for engraftment human stem cells, e.g. into immune deficient mice such as irradiated beige, nude, Xid (X-linked immune deficiency), SCID and non-obese diabetic SCID (NOD/SCID) mice, and in utero transplantation into sheep fetuses which resulted in successful multilineage engraftment of both myeloid and lymphoid cells.

Previously inventors have developed a functional in vivo assay primitive human SCID repopulating cells (SRCs) based on their ability to durably repopulate the bone marrow of intravenously transplanted SCID or NOD/SCID mice with high levels of both myeloid and lymphoid cells ([1, 2]). Kinetic experiments demonstrated that only a small fraction of the transplanted cells engrafted and that these cells repopulated the murine bone marrow by extensive proliferation and differentiation. Furthermore, the primitive human cells also retained the capacity to engraft secondary murine recipients [3]. Transplantation of populations enriched for CD34 and CD38cell surface antigen expression, revealed that the phenotype of SRC is CD34+CD38− [2]. Other repopulating cells may exist since recent studies suggest that immature human CD34− cells and more differentiated CD34+CD38+ cells have some limited engraftment potential [4, 5].

Accumulating evidence indicates that stem cell homing to the bone marrow is a multistep process. The mechanisms involved in hematopoietic stem cell trafficking have been largely unknown for a long time.

During the past few years, the role of particular secreted (eg, cytokines) and cell-bound proteins (eg, adhesion molecules) in progenitor mobilization and homing has been recognized.[6-9] More recently, it has been shown that cytokines may play a central role in progenitor cell trafficking, particularly in stem cell homing to the bone marrow (BM) [9-12]. Interestingly, extravasation of mature leukocytes during inflammation and homing of immature progenitor and stem cells to the BM may at least partially depend on similar mechanisms [8]. Inflamed tissues and the hematopoietic microenvironment share similarities, such as expression of particular adhesion molecules (E-selectin, vascular cell adhesion molecule-1) on microvascular endothelium [13, 14].

Of particular interest for bone marrow engraftment are the chemokine stromal cell-derived factor-1 (SDF-1) and its receptor CXCR4. Treatment of human progenitor cells with antibodies to CXCR4 prevented engraftment into human severe combined immunodeficient (NOD/SCID) mice. In vitro CXCR4− dependent migration to SDF-1 of CD34+CD38−/low cells was found to correlate with. in vivo engraftment and stem cell function [10]. Activation of CD34(+) cells with SDF-1α leads to firm adhesion and transendothelial migration, which is dependent on LFA-1/ICAM-1 (intracellular adhesion molecule-1) and VLA-4/VCAM-1 (vascular adhesion molecule-1). Furthermore, SDF-1-induced polarization and extravasation of CD34(+)/CXCR4(+) cells through the extracellular matrix underlining the endothelium is dependent on both VLA-4 and VLA-5[15].

In view of expanded approach to treatment of many severe diseases by hematopoietic stem cell transplantation, it is highly desirable to understand better the mechanism behind stem cell homing to the bone marrow and repopulation of transplanted hosts in order to obtain stem cells with higher rates of successful and long-term engraftment.

### SUMMARY OF THE INVENTION

The present invention is concerned with a new function of the chemokine HCC-1. It has now been found, according to the present invention, that treatment of the murine hematopoietic FDCP-Mix progenitor cells with HCC-1, glycosylated HCC-1 and N-terminally truncated HCC-1 molecules induce a chemotactic migration. In this context glycoslylated HCC-1 was identified in a screening for chemotactic activities with subsequent purification of glycosylated HCC-1 from human blood filtrate.

Furthermore in an in vivo transplant model using irradiated mice it was found that pretreatment (priming) of mononuclear cells containing murine stem cells with HCC-1 improves stem cell engraftment in the bone marrow.

Furthermore it was found that priming of human cord blood mononuclear cells containing human hematopoletic progenitor cells with HCC-1 prior to transplantation improves engraftment in the bone marrow of sublethally irradiated NOD/SCID mice.

The present invention thus relates to a method increasing the engraftment of hematopoietic stem and progenitor cells for use in clinical transplantation. The method is related to a pretreatment (priming) of transplantable hematopoietic progenitor- and stem cells with HCC-1 prior to transplantation and/or to in vivo application of HCC-1 to patients prior-, during, and/or subsequently to stem cell transplantation.

A further aspect of the invention relates to a method for transplantation of immature hematopoietic cells in patients. The patients need conditioning under sublethal, lethal or supralethal conditions, for example by total body irradiation (TBI) and/or by treatment with myeloablative and immunosupressive agents according to standard protocols. For example, a sublethal dose of irradiation is within the range of 3-7 Gy TBI, a lethal dose is within the range of 7-9.5 Gy TBI, and a supralethal dose is within the range of 9-16.5 Gy TBI. Examples of myeloablative agents are busulphan, dimethyl mileran and thiotepa, and of immunosupressive agents are prednisolone, methyl prednisolone, azathioprine, cyclophosphamide, cyclophosphamide, etc.

The method of the invention is suitable for the treatment of diseases curable by bone marrow transplantation such as malignant diseases, including leukemias, solid tumors, congenital or genetically-determined hematopoietic abnormalities, like severe combined immunodeficiency syndromes (SCID) including adenosine deaminase (ADA) deficiency, osteopetrosis, aplastic anemia, Gaucher's disease, thalassemia.

### BRIEF DESCRIPTION OF THE DRAWINGS

FIG. 1: Purification step A: Reverse phase chromatography, using a Bakerbond cartrige (47 mm i.d.×300 mm) with an acetonitril gradient.

FIG. 2: Purification step B: Size exclusion chromatography using SEC Superdex 16/60 High Load column with the eluent PBS, pH 7.4.

FIG. 3: Purification step C: Reverse phase chromatography, using a YMC C18 (10 mm i.d.×250 mm) with an acetonitril gradient.

FIG. 4: Purification step D: Reverse phase chromatography, using a YMC C18 (4 mm i.d.×250 mm) with an acetonitril gradient.

FIG. 5: Chemotactic activity of HCC-1 (1-74) and glycosylated HCC-1 (1-74) on FDCP-Mix cells.

FIG. 6: Chemotactic activity of HCC-1 (1-74) and HCC-1 (9-74) on FDCP-Mix cells.

FIG. 7: Concept of the modulation of homing mechanisms by preincubation with HCC-1.

FIG. 8: Validation of the potential of HCC-1 (1-74) to increase the adhesion of FDCP-mix progenitor cells to HUVEC endothelial cells under a shear stress of 2 dynes/cm². The experiment was performed with and without (control) preincubation of progenitor cells with HCC-1 (1-74). Increased adhesion of hematopoietic FDCP-Mix progenitor cells HCC-1 to endothelium due to priming of the cells with HCC-1 was observed.

FIG. 9: Priming of murine hematopoietic progenitor cells with HCC-1 causes an increased engraftment of the cells in the murine competitive repopulation model. The potential of HCC-1 (1-74) to improve engraftment of murine progenitor cells in the bone marrow was tested using competitive engraftment of Ly5.1 cells against Ly5.2 cells after preincubation of Ly5.1 donor cells. The cells were pre-incubated with HCC-1 (1-74) with a concentration of 1000 ng/ml. Transplantation was performed into sublethally irradiated C57BL6/Ly5.2 mice. The percentage of Ly5.1 donor cells was determined 5 weeks after transplantation in the bone marrow. The results of 4 experiments were pooled. Mean is shown as bar, result from a single animal is shown as dot.

FIG. 10: Priming of human hematopoietic progenitor cells with HCC-1 causes an increased engraftment of the cells in NOD/SCID mice. For the priming procedure cells were incubated for 30 min with HCC-1. Subsequently the peptide was removed by washing the cells. The cells were injected i.v. into sublethally irradiated NOD/SCID mice. After 8 weeks animals were killed and the percentage of human HLA-I⁺/CD33⁺ cells was detected in the bone marrow by FACS analysis using human specific antibodies (A) For priming of cells HCC-1 (1-74) was used in a concentration of 1000 ng/ml. The results of two experiments were pooled and normalized to the mean of the control. (Number of animals: control=9, experimental group 8; p=0.057) (B) For priming of the cells with HCC-1 (9-74) different concentrations are used. For each HCC-1 (9-74) concentration 8 animals were tested. In the experimental group of 100 ng/ml and 1000 ng/ml a significant increase of engrafted human HLA-I⁺/CD33⁺ cells was detected (p<0.05). Mean is shown as bar, result from a single animal is shown as dot.

### DETAILED DESCRIPTION OF THE INVENTION

The present invention concerns a poly peptide having at least 90% homology with the amino acid sequence 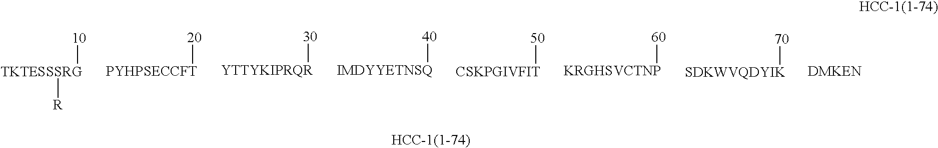
whereby R is an oligosaccharide composed out of N-acetylgalactosamine galactose or an oligosacharide composed out of N-acetylgalactosamine galactose and N-acetylneuraminic acids

Its biologically active fragments, analogs and derivatives, in particular amidated, acylated, and/or phosphorylized derivatives wherein the two cystein residues in positions 16 and 40 linked together by a disulfide bond and wherein the two cystein residues in positions 17 and 56 are linked together by a disulfide bond.

In the context of the description of the invention the term “homology” means identical amino acids in an amino acid sequence, as well as amino acids which are modified without altering the function of the molecule. Also amino acids may be substituted in the polypeptide chain which amino acids are conservatively exchanged amino acids. Such amino acids are e.g. neutral amino acids, aromatic amino acids charged amino acids and the like. For example an exchange of serine against valine or lysine against asparagine may not alter the function of the polypeptide of the invention.

In particular, a polypeptide of the invention has at least 90% identity to the polypeptide sequence of the invention.

The polypeptide of the invention is in particular the glycosylated chemokine HCC-1. The processed chemokine of the invention comprises a polypeptide wherein (a) the N-terminus is modified by coupling a chemical group generating a chemokine having the structure of [Glyoxyloyl1]PHC 1-Pentane oxime, Nonanyl-PHC, [Glyoxyloyl1]PHC 1-Heptane oxime, [Glyoxyloyl1]PHC 1-Hexane oxime, [Glyoxyloyl1]PHC 1-Pentene oxime or Nonaoyl-PHC and wherein the modification is influencing the biological activity of PHC or (b) wherein amino acid residues of the N-terminus or of the C-terminus are deleted.

The polypeptides, of the invention comprise modifications which are Increasing the plasma half-life time of HCC-1 which by way of example may be achieved by introducing one or more lysine, histidine, glutamate, aspartate, or cysteine residues which are e. g. modified by coupling a chemical group having the structure of poly ethylene glycol.

Subject-matter of the invention is also an antibody against an amino acid sequence of the invention. The skilled person knows very well how to obtain antibodies against a polypeptide by immunizing e.g. animal with the respective polypeptide. From polyclonal antibodies monoclonals may be derived by established methods based on clonal selection techniques. Polyclonal or monoclonal antibodies against the polypeptide such as chemokine HCC-1 of the invention may serve as starting material for diagnostic agents or may be used directly for detecting the level of the polypeptide of the invention.

From the polypeptide and/or the antibody of the invention a medicament can be manufactured. The skilled person knows very well how to provide an appropriate galenic preparation.

A process for producing a polypeptide is also subject matter of the invention. The polypeptide of the invention can be manufactured using recombinant techniques or chemical synthesis.

The polypeptides of the invention may also be manufactured by utilizing the cellular expression system. A process for producing cells capable of expressing a polypeptide of the invention is also subject-matter of the invention.

According to the invention the polypeptide of the invention e.g. HCC-1, HCC-1 molecules without glycosylation and N-terminally truncated HCC-1 molecules, especially HCC-1 (2-74), HCC-1 (3-74), HCC-1 (4-74), HCC-1 (5-74), HCC-1 (6-74), HCC-1 (7-74), HCC-1 (8-74), HCC-1 (9-74), HCC-1 (10-74), HCC-1 (11-74) and HCC-1 (12-74) can be used to increase engraftment of stem cells, for transplantation of progenitor and stem cells, for treatment of progenitor-and stem cells prior to transplantation, for in vivo application of such a molecule into patients which are receiving stem cell transplantation prior to and/or in the course of stem cell transplantation. This is in particular useful, if the host patient is not conditioned or conditioned e. g. under sublethal, lethal, or supralethal conditions. Sublethal, lethal, or supralethal conditions include treatment with total body irradiation, optionally followed by treatment with myeloablative or immunosuppressive agents; myeloablative or immunosuppressive treatment without total body irradiation.

Furthermore, the polypeptide of the invention can be used for the transplantation of hematopoietic progenitor and stem cells, umbilical cord blood and placental stem and progenitor cells, liver stem and progenitor cells (oval cells), mesenchymal stem and progenitor cells, endothelial progenitor cells, skeletal muscle stem and progenitor cells (satellite cells), smooth muscle stem and progenitor cells, intestinal stem and progenitor cells, embryonic stem cells, and genetically modified embryonic stem cells, adult islet/beta stem- and progenitor cell, epidermal progenitor and stem cells, keratinocyte stem cells of cornea, skin and hair follicles, olfactory (bulb) stem and progenitor cells and side population cells from diverse adult tissues.

The polypeptide of the invention may be used as well for the treatment of leukemias, lymphoproliferative disorders, aplastic anemia, congenital disorders of the bone marrow, solid tumors, autoimmune disorders, inflammatory diseases, primary immunodeficiencies, primary systemic amyloidosis, systemic sclerosis, heart diseases, liver diseases, neurodegenerative diseases, multiple sclerosis, M. Parkinson, stroke, spinal cord injury diabetes mellitus, bone diseases, skin diseases, replacement therapy of the skin, retina or cornea, other congenital disorders, vessel diseases like atherosclerosis or cardiovascular disease.

The invention is described by the following non-limiting examples.

### EXAMPLE 1

### Identification of Glycosylated HCC-1 as a Stem Cell Migrating Activity

900 L of human hemofiltrate (HF) for large scale recovery of plasma peptides were obtained from chemotherapy-treated patients with renal failure. Ultrafilters used for hemofiltration had a specified molecular mass cut-off of 20 kD. The sterile filtrate was immediately cooled to 4° C. and acidified to pH 3 to prevent bacterial growth and proteolysis. For peptide extraction the HF was ultrafiltrated a second time. The filtrate was conditioned to pH 2.7 and applied onto the strong cation exchanger, Fractogel TSK SP 650(M), 100×250 mm (Merck, Darmstadt, Germany) using an Autopilot chromatography system (PerSeptive Biosystems, Wiesbaden, Germany). Bound peptides were eluted using seven buffers with increasing pH resulting in seven pH-pools. The seven buffers were composed as follows: I: 0.1 M citric acid monohydrate, pH 3.6; II: 0.1 M acetic acid +0.1 M sodium acetate, pH 4.5; III: 0.1 M malic acid, pH 5.0; IV: 0.1 M succinic acid, pH 5.6; V: 0.1 M sodium dihydrogen phosphate, pH 6.6; VI: :0.1 M disodiumhydrogen phosphate, pH 7.4; VII: 0.1 M ammonium carbonate, pH 9.0. The seven pools (pH pools) were collected and each of them was loaded onto a RP column, 125 mm×100 mm i.d., Source RPC, 15 μm (Pharmacia) and eluted in a gradient from 100% A (0.01 M HCl in water) to 60% B (0.01 M HCl in 80% acetonitrile). Fractions of 200 mL were collected. In the screening for chemotactic activities using the FDCP-Mix stem cell line the predominant activity was identified in pH pool VI. This chemotactic activity was purified in four further chromatographic steps A to D. (A) Reverse phase chromatography, using a Bakerbond cartrige (47 mm i.d.×300 mm) with an acetonitril gradient. (B) Size exclusion chromatography using SEC Superdex 16/60 High Load column with the eluent PBS, pH 7.4. (C) Reverse phase chromatography, using a YMC C18 (10 mm i.d.×250 mm) with an acetonitril gradient. (D) Reverse phase chromatography, using a YMC C18 (4 mm i.d.×250 mm) with an acetonitril gradient.

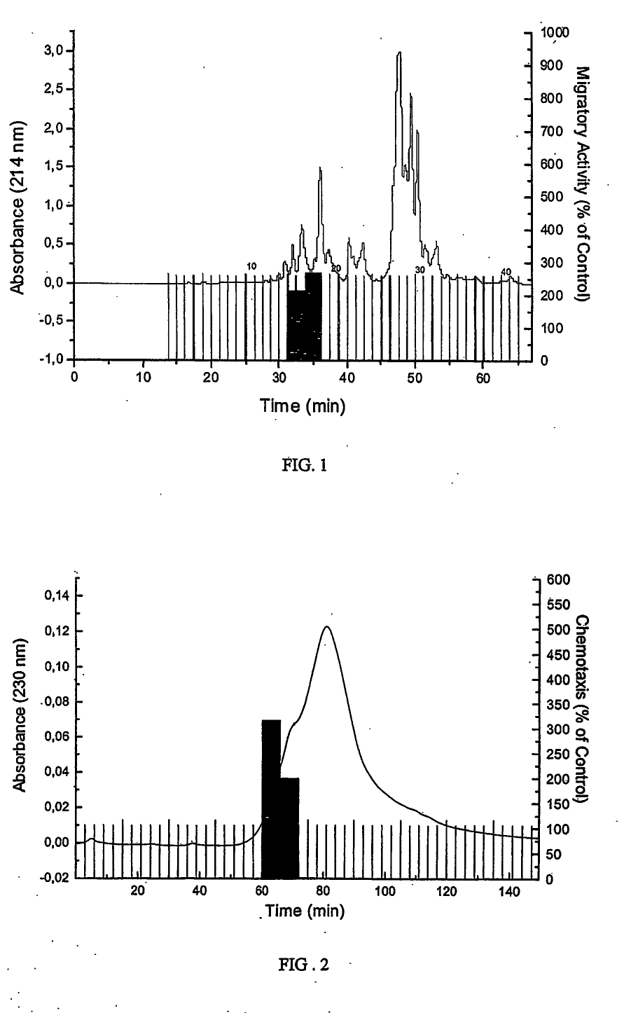

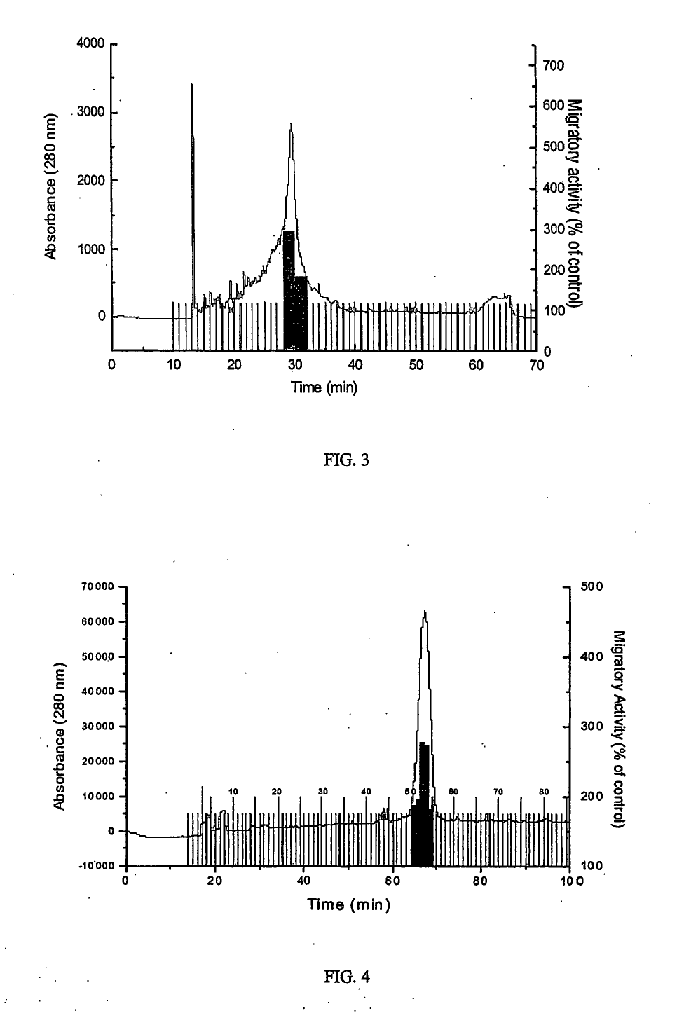

Amino acid sequencing by Edman degradation of the purified material revealed the sequence of HCC-1. Mass spectrometric analysis of the isolated material revealed a glycosylated molecule. The isolated molecules revealed molecular weights (Mw) of 9038.15 and 9331.9. Whereas HCC-1 (1-74) carries Mw of 8673.09 the increase of the Mw in the isolated molecules was identified as an O-glycosylation of the amino acid Serine in position 7 with N-acetylgalactosamine galactose and with oligosacharide composed of N-acetylgalactosamine galactose and N-acetylneuraminic acid. FIG. 1 shows the purification step A: Reverse phase chromatography, using a Bakerbond cartrige (47 mm i.d.×300 mm) with an acetonitril gradient. FIG. 2 shows the purification step B: Size exclusion chromatography using SEC Superdex 16/60 High Load column with the eluent PBS, pH 7.4. FIG. 3 shows the purification step C: Reverse phase chromatography, using a YMC C18 (10 mm i.d.×250 mm) with an acetonitril gradient. FIG. 4 shows the purification step D: Reverse phase chromatography, using a YMC C18 (4 mm i.d.×250 mm) with an acetonitril gradient.

### EXAMPLE 2

### Chemotactic Activity of HCC-1 Molecules to the Murine FDCP-Mix Stem Cell Line

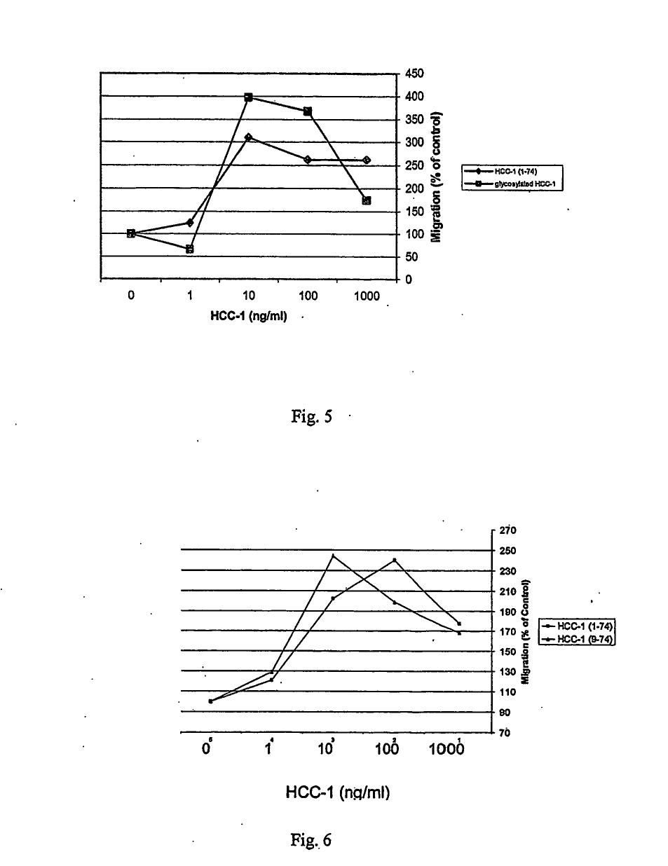

FIGS. 5 and 6 are showing FDCP-Mix cells which were subjected to in vitro chemotactic assays. Chemotaxis was assessed in 96-transwell chambers (Neuroprobe, Cabin John, MD) by using polyvinylpyrrolidone-free polycarbonate membranes (Nucleopore, Neuroprobe) with 5-μm pores. Four hundred microliters of IMDM medium was added to the bottom of the well, and was supplemented with varying concentrations of HCC-1 molecules. 200 μl of IMDM medium containing 100.000 FDCP-Mix cells were added to the upper wells of the chemotaxis chamber. All assays were carried out in triplicate, and the migrated cells were counted in 4 randomly selected fields at 63-fold magnification after migration for 14 h.

### EXAMPLE 3

### Modulation of Homing Mechanisms by Preincubation with HCC-1 In Vitro

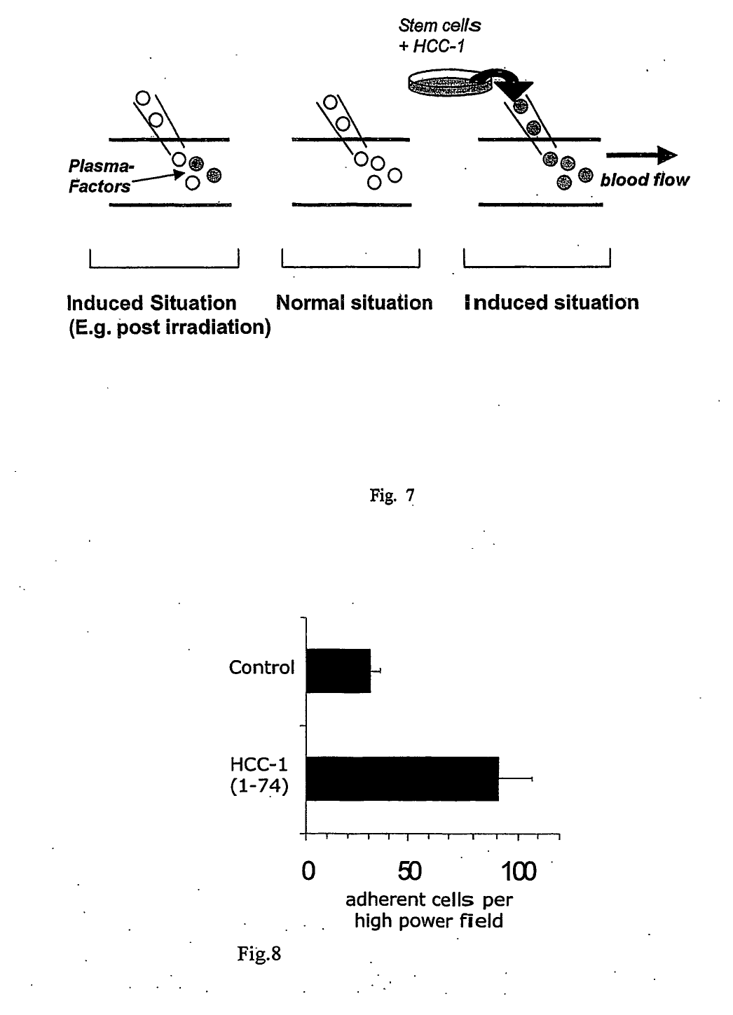

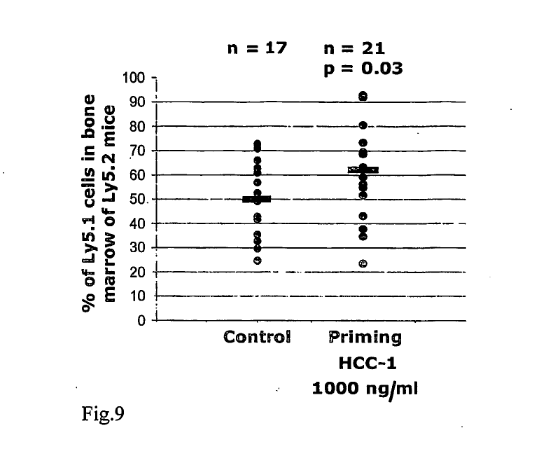

Enriched Mononulcear cells, CD34+ progenitor cells from human cord blood, mobilized peripheral blood, or bone marrow are incubated with HCC-1 in concentrations between 100 pM and 10 μM for a time period which is between 5 minutes and 12 hours. FIG. 7 describes the concept of the modulation of homing mechanisms by preincubation with HCC-1.

After preincubation stem cells are transplanted into the blood flow. In a competitive repopulation model using Ly 5.1 and Ly 5.2 mice it was shown that preincubation of the cells gives an advantage in the engraftment of the bone marrow over cells which were not treated with HCC-1.

### EXAMPLE 4

### Validation Of HCC-1 in the Adhesion Assay

The potential of HCC-1 (1-74) to increase the adhesion of FDCP-mix progenitor cells to HUVEC endothelial cells under a shear stress of 2 dynes/cm² was validated. With and without preincubation of progenitor cells with HCC-1 (1-74). The chemokine HCC-1 (1-74) was shown to improve the adhesion of hematopoietic progenitor cells to the endothelium. Cells from the hematopoietic progenitor cell line (FDCP-Mix) were primed with 1000 ng/ml HCC-1 (1-74) and subsequently injected into a flow chamber. Adhesion of the progenitor cells to endothelial cells was detected under a shear stress of 2 dynes/cm².

### EXAMPLE 5

### Validation in the Competitive Repopulation Model

In the competitive Ly 5.1/Ly 5.2 repopulation model Ly5.1 bone marrow cells were preincubated with 1000 ng/ml HCC-1 (1-74). Subsequently the cells were washed, mixed in a ratio of 50:50 with Ly5.2 bone marrow cells, and injected i.v. into sublethally irradiated Ly5.2 mice. After 5 weeks animals were killed and the amount of Ly5.1 cells in the bone marrow was detected. The results show that preincubation with HCC-1 induces a significant increase of the engraftment of the Ly5.1 cells (p=0.03) (FIG. 9).

### EXAMPLE 6

### Validation in the NOD/SCID Mouse Model

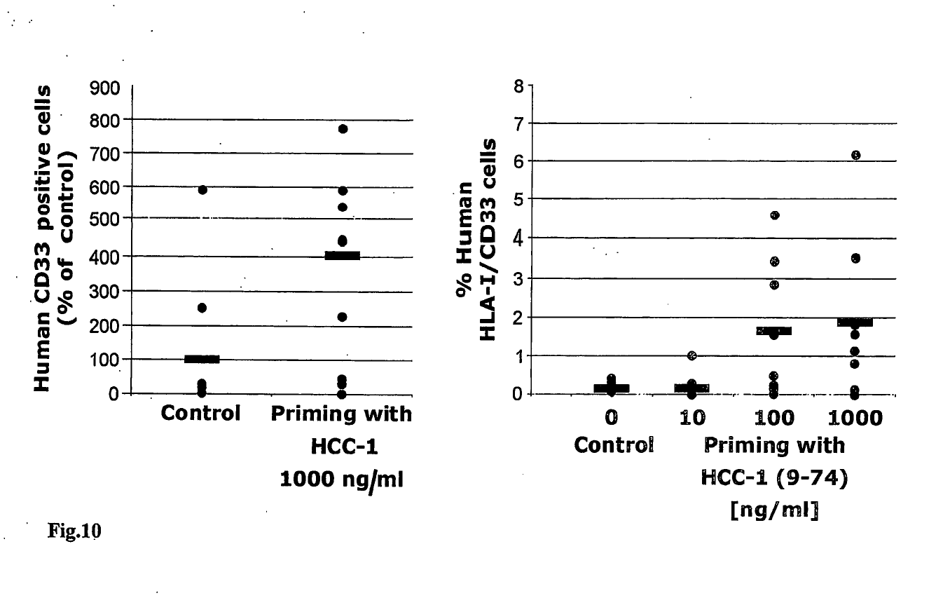

It was investigated, if the priming of human progenitor cells with HCC-1 for 30 min increases the engraftment of the cells in Non-Obese Diabetic/Severe Combined Immunodeficiency (NOD/SCID) mice (FIG. 10). In these experiments HCC-1 (1-74) as well as HCC-1 (9-74) were tested. By priming of the cells with HCC-1 (1-74) an increase of the engraftment could be achieved.

The priming with HCC-1 (9-74) showed a dose dependent increase of the engraftment. The highest concentrations of 100 ng/ml and 1000 ng/ml resulted in a significant increase of the engraftment

### REFERENCES

1. Lapidot T, P. F., Doedens M, Murdoch B, Williams D E, Dick J E, Cytokine stimulation of multilineage hematopoiesis from immature human cells engrafted in SCID mice. Science, 1992. 255: p. 255.
2. Larochelle A, V. J., Hanenberg H, Wang J C, Bhatia M, Lapidot T, Moritz T, Murdoch B, Xiao X L, Kato I, Williams D A, Dick J E, Identification of primitive human hematopoietic cells capable of repopulating NOD/SCID mouse bone marrow: implications for gene therapy. Nat Med, 1996. 2: p. 1329-37.
3. Cashman J, B. K., Hogge D E, Eaves A C, Eaves C J, Sustained proliferation, multi-lineage differentiation and maintenance of primitive human haemopoietic cells in NOD/SCID mice transplanted with human cord blood. Br J Haematol, 1997. 98: p. 1026-36.
4. Zanjani E D, A.-P. G., Livingston A G, Flake A W, Ogawa M, Human bone marrow CD34− cells engraft in vivo and undergo multilineage expression that includes giving rise to CD34+ cells. Exp Hematol, 1998. 26: p. 353-60.
5. Conneally E, C. J., Petzer A, Eaves C, Expansion in vitro of transplantable human cord blood stem cells demonstrated using a quantitative assay of their lympho-myeloid repopulating activity in nonobese diabetic-scid/scid mice. Proc Natl Acad Sci USA, 1997. 94: p. 9836-41.
6. Esmail D. Zanjani, A. W. F., Graca Almeida-Porada, Nam Tran, and Thalia Papayannopoulou, Homing of Human Cells in the Fetal Sheep Model: Modulation by Antibodies Activating or Inhibiting Very Late Activation Antigen-4-Dependent Function. Blood, 1999. 94: p. 2515-2522.
7. Greenberg A W, K. W., Hammer D A, Relationship between selectin-mediated rolling of hematopoietic stem and progenitor cells and progression in hematopoietic development. Blood, 2000. 95: p. 478-86.
8. Mohle R, B. F., Rafil S, Moore M A, Brugger W, Kanz L, Regulation of transendothelial migration of hematopoietic progenitor cells. Ann N Y Acad Sci, 1999. 872: p. 176-85.
9. Naiyer A J, J. D., Ahn J, Mohle R, Peichev M, Lam G, Silverstein R L, Moore M A, Rafii S, Stromal derived factor-1-induced chemokinesis of cord blood CD34(+) cells (long-term culture-initiating cells) through endothelial cells is mediated by E-selectin. Blood, 1999. 94: p. 4011-9.
10. Peled A, P. I., Kollet O, Magid M, Ponomaryov T, Byk T, Nagler A, Ben-Hur H, Many A, Shultz L, Lider O, Alon R, Zipori D, Lapidot T, Dependence of human stem cell engraftment and repopulation of NOD/SCID mice on CXCR4. Science, 1999. 283: p. 845-8.
11. Aiuti A, W. I., Bleul C, Springer T, Gutierrez-Ramos J C, The chemokine SDF-1 is a chemoattractant for human CD34+ hematopoietic progenitor cells and provides a new mechanism to explain the mobilization of CD34+ progenitors to peripheral blood. J Exp Med, 1997. 185: p. 111-20.
12. Mohle R, B. F., Rafii S, Moore M A, Brugger W, Kanz L, The chemokine receptor CXCR-4 is expressed on CD34+ hematopoietic progenitors and leukemic cells and mediates transendothelial migration induced by stromal cell-derived factor-1. Blood, 1998. 91: p. 4523-30.
13. Schweitzer K M, D. A., van der Valk P, Thijsen S F, Zevenbergen A, Theijsmeijer A P, van der Schoot C E, Langenhuijsen M M, Constitutive expression of E-selectin-and vascular cell adhesion molecule-1 on endothelial cells of hematopoietic tissues, Am J Pathol, 1996. 148: p. 165-75.
14. Jacobsen K, K. J., Kincade P W, Osmond D G, Adhesion receptors on bone marrow stromal cells: in vivo expression of vascular cell adhesion molecule-1 by reticular cells and sinusoidal endothelium in normal and gamma-irradiated mice. Blood, 1996. 87: p. 73-82.
15. Peled A, K. O., Ponomaryov T, Petit I, Franitza S, Grabovsky V, Slav M M, Nagler A, Lider O, Alon R, Zipori D, Lapidot T, The chemokihe SDF-1 activates the integrins LFA-1, VLA-4, and VLA-5 on immature human CD34(+) cells: role in transendothelial/stromal migration and engraftment of NOD/SCID mice. Blood, 2000. 95: p. 3289-96.

1. A polypeptide having at least 90% homology with the amino acid sequence 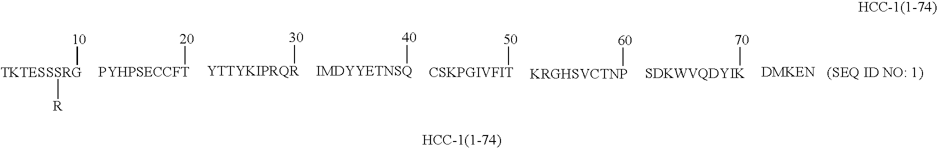
whereby R is an oligosaccharide composed out of N-acetylgalactosamine galactose or an oligosacharide composed out of N-acetylgalactosamine galactose and N-acetylneuraminic acids, its biologically active fragments, analogs and derivatives, in particular amidated, acylated, and/or phosphorylized derivatives,
wherein the two cystein residues in positions 16 and 40 linked together by a disulfide bond and wherein the two cystein residues in positions 17 and 56 are linked together by a disulfide bond.

2. A polypeptide having at least 90% identity to the polypeptide sequence of claim 1.

3. The polypeptide of claim 1 being the chemokine glycosylated HCC-1.

4. The processed polypeptide according to claim 1 wherein (a) the N-terminus is modified by coupling a chemical group generating a chemokine having the structure of [Glyoxyloyl1]PHC 1-Pentane oxime, Nonanyl-PHC, [Glyoxyloyl1]PHC 1-Heptane oxime, [Glyoxyloyl1]PHC 1-Hexane oxime, [Glyoxyloyl1]PHC 1-Pentene oxime or Nonaoyl-PHC and wherein the modification is influencing the biological activity of PHC or wherein (b) amino acid residues of the N-terminus or of the C-terminus are deleted.

5. The chemokines according to claim 1 wherein one or more lysine, histidine, glutamate, aspartate, or cysteine residues of the chemokine are modified by coupling a chemical group having the structure of polyethylenglycol and wherein this modification is increasing the plasma half-life time of HCC-1.

6. An antibody against an amino acid sequence of claim 1.

7. A diagnostic agent containing polyclonal or monoclonal antibodies against chemokine HCC-1 of claim 1.

8. A medicament containing chemokine HCC-1 of claim 1 or an antibody against the chemokine.

9. A process for producing a polypeptide comprising polypeptides according to claim 1 using recombinant techniques or chemical synthesis.

10. A process for producing cells capable of expressing a polypeptide according to claim 1.

11. Use of the polypeptide according to claim 1, in particular HCC-1 molecules without glycosylation and N-terminally truncated HCC-1 molecules, especially HCC-1 (2-74), HCC-1 (3-74), HCC-1 (4-74), HCC-1 (5-74), HCC-1 (6-74), HCC-1 (7-74), HCC-1 (8-74), HCC-1 (9-74), HCC- 1 (10-74), HCC-1 (11-74) and HCC-1 (12-74) for the manufacturing of a medicament for increase engraftment of stem cells.

12. Use of the polypeptide according to claim 11 for transplantation of progenitor and stem cells.

13. Use of the polypeptide according to claim 11 for treatment of progenitor- and stem cells prior to transplantation.

14. Use of the polypeptide according to claim 11 for in vivo application of such a molecule into patients which are receiving stem cell transplantation prior to and/or in the course of stem cell transplantation.

15. Use of the polypeptide according to claim 14 wherein the host patient are not conditioned.

16. Use of the polypeptide according to claim 14 wherein the host patient is conditioned under sublethal, lethal, or supralethal conditions.

17. Use of the polypeptide according to claim 16 wherein sublethal, lethal, or supralethal conditions include treatment with total body irradiation, optionally followed by treatment with myeloablative or immunosuppressive agents.

18. Use of the polypeptide according to claim 16 wherein sublethal, lethal, or supralethal conditions include myeloablative or immunosuppressive treatment without total body irradiation.

19. Use of the polypeptide according to claim 11 for the transplantation of hematopoietic progenitor and stem cells, umbilical cord blood and placental stem and progenitor cells, liver stem and progenitor cells (oval cells), mesenchymal stem and progenitor cells, endothelial progenitor cells, skeletal muscle stem and progenitor cells (satellite cells), smooth muscle stem and progenitor cells, intestinal stem and progenitor cells, embryonic stem cells, and genetically modified embryonic stem cells, adult islet/beta stem- and progenitor cell, epidermal progenitor and stem cells, keratinocyte stem cells of cornea, skin and hair follicles, olfactory (bulb) stem and progenitor cells and side population cells from diverse adult tissues.

20. Use of the polypeptide according to claim 11 for the treatment of leukemias, lymphoproliferative disorders, aplastic anemia, congenital disorders of the bone marrow, solid tumors, autoimmune disorders, inflammatory diseases, primary immunodeficiencies, primary systemic amyloidosis, systemic sclerosis, heart diseases, liver diseases, neurodegenerative diseases, multiple sclerosis, M. Parkinson, stroke, spinal cord injury diabetes mellitus, bone diseases, skin diseases, replacement therapy of the skin, retina or cornea, other congenital disorders, vessel diseases like atherosclerosis or cardiovascular disease.
